(19) United States
(12) Patent Application Publication
(10) Pub. No.: US 2023/0058740 A1
(43) Pub. Date: Feb. 23, 2023

(54) Robust Protein Expression Enabled by Dynamic Control over Host Proteases

(71) Applicant: Duke University, Durham, NC (US)

(72) Inventors: Michael Lynch, Durham, NC (US); Romel Menacho Melgar, Durham, NC (US)

(21) Appl. No.: 17/819,100

(22) Filed: Aug. 11, 2022

Related U.S. Application Data
(60) Provisional application No. 63/232,433, filed on Aug. 12, 2021.

(51) Int. Cl. C12N 15/70

(52) U.S. Cl. CPC C12N 15/70

(57) ABSTRACT

Provide herein are engineered microbial strains having greatly improved expression of many challenging protein product. This system relies on controlled expression or dynamic reduction in activity of key housekeeping proteases. Dynamic control, implemented with CRISPR based gene silencing and or controlled protein degradation enables increased protein expression while minimizing the negative impact of complete deletions of these housekeeping enzymes.

### FEDERALLY SPONSORED RESEARCH OR DEVELOPMENT

This invention was made with government support under NCBC #2018BIG6503; DOE #EE7563; and NIH # R61AI140485-01. The government has certain rights in the invention.

### FIELD OF THE INVENTION

This invention relates to metabolically engineered microorganisms, such as bacterial strains, in which regulation of proteases results in improved production of a protein.

### BACKGROUND OF THE INVENTION

Recombinant protein expression is critical in many aspects of basic and applied research in numerous areas of biotechnology. Additionally, together industrial and clinical proteins comprise a growing multi-billion dollar annual global market. Numerous efforts over the past several decades have been focused on engineering improved production hosts for both specific proteins as well as proteins in general, including in E. coli, a mainstay for protein expression. Despite many advances and the ease of many cloning and expression protocols, there remain numerous proteins that are difficult to express in E. coli. As a result, significant effort can be spent optimizing expression protocols, such as medium, induction time, level and temperature to enable adequate expression. In many cases, it is often easier to try to engineer the protein or to give up on E. coli and screen several additional expression hosts such as Bacillus Saccharomyces, Streptomyces, or mammalian cell lines such as Chinese hamster ovary (CHO) or human embryonic kidney (HEK), or even in vitro cell free expression. While these additional expression systems have their own advantages and disadvantages, and are not conceptually more complicated than expression in E. coli, they can add significant cost and time to obtaining purified protein. These alternative hosts are often impractical for a lab without prior experience and can necessitate significant investment in developing working expertise with a new expression platform. Improved E. coli hosts that overcome challenges with “hard to express” proteins are of use to many labs working with alternative expression systems.

While it can be difficult to define a “hard to express” protein, for the sake of this discussion we will define a few classes of proteins that have proven difficult in E. coli: toxic proteins, proteins that are slow to fold, and large proteins. Toxic protein can be defined as those whose induction causes significant growth arrest leading to reduced growth and expression. Proteins that are slow to fold, may normally rely on chaperones or cofactors for rapid folding in native hosts, and when expressed in E. coli may lead to partially folded states that are recognized and degraded by E. coli proteases prior to maturation. Large proteins as the name implies are proteins nominally larger than 100 kDa that can be difficult to express, and may make up a special class of slow folders, wherein protein size alone reduces rates of maturation, or where other structures may need to recognition and unfolding and or degradation. Large proteins are of particular interest in the fields of natural products wherein many polyketide synthases and non-ribosomal peptide synthases are large and can be difficult to express in E. coli.

### SUMMARY OF THE INVENTION

We describe the development of engineered microbial strains offering greatly improved expression of many challenging proteins. This system relies on controlled expression of key housekeeping proteases to enable increased protein expression.

Other methods, features and/or advantages is, or will become, apparent upon examination of the following Figures and detailed description. It is intended that all such additional methods, features, and advantages be included within this description and be protected by the accompanying claims.

### BRIEF DESCRIPTION OF THE DRAWINGS

The novel features of the invention are set forth with particularity in the claims. A better understanding of the features and advantages of the present invention will be obtained by reference to the following detailed description that sets forth illustrative embodiments, in which the principles of the invention are used, and the accompanying drawings of which:

FIG. 1A-1B depicts two-stage dynamic control of host machinery such as proteases.

FIG. 2A-2C depicts graphs demonstrating further two-stage dynamic control.

FIG. 3A-3E depicts two-stage dynamic control of Lon protease activity.

FIG. 4 depicts two-stage dynamic control of FtsH protease activity.

FIG. 5A-5B depicts improved protein production with two-stage dynamic control over host proteases.

### DETAILED DESCRIPTION OF THE INVENTION

Despite being a routine workflow in many biology laboratories, routine recombinant protein expression in E. coli can often prove challenging, specifically when dealing with ill-defined “difficult” or “hard to express” proteins. We report the development of engineered strains of E. coli offering greatly improved expression of many challenging proteins. This system relies on tightly controlled expression, which is auto-induced in a phosphate depleted stationary phase, coupled with the deletion of, or dynamic reduction in activity of, key housekeeping proteases including HslUV, FtsH and Lon. Dynamic control, implemented with CRISPR based gene silencing and or controlled protein degradation enables increased protein expression while minimizing the negative impact of complete deletions of these housekeeping enzymes.

Most studies looking at protein expression have been complicated by induction of heterologous protein expression during the growth phase, such as in mid-exponential phase. While this traditional methodology would seem to leverage a highly productive cellular state for protein synthesis it leads to competition between cellular growth and heterologous production as well as potentially to cellular toxicity and decreased growth rate. We have recently demonstrated the tightly controlled production of heterologous proteins in stationary phase, where expression is auto-induced by phosphate depletion, leading to relatively high protein titers. These initial results confirm the utility of stationary phase expression. In addition, in these studies we evaluated the relative impact of Lon protease activity on the expression of a Lon substrate, when two stage expression was compared to expression using BL21(DE3) a known Lon deficient strain. Although we did observe expression of Lon substrates, the levels were far from optimal when compared to non-Lon substrate controls. These results indicated that there was room to further reduce the activity of Lon, and protease activity more generally, to improve the expression of heterologous proteins that may be substrates of native housekeeping proteases. In the examples, protease activity is addressed in detail.

### Definitions

As used in the specification and the claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “microorganism” includes a single microorganism as well as a plurality of microorganisms; and the like.

The term “heterologous DNA,” “heterologous nucleic acid sequence,” and the like as used herein refers to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid, such as a nonnative promoter driving gene expression.

The term “synthetic metabolic valve,” and the like as used herein refers to either the use of controlled proteolysis, gene silencing or the combination of both proteolysis and gene silencing to alter metabolic fluxes.

The term “heterologous” is intended to include the term “exogenous” as the latter term is generally used in the art. With reference to the host microorganism's genome prior to the introduction of a heterologous nucleic acid sequence, the nucleic acid sequence that codes for the enzyme is heterologous (whether or not the heterologous nucleic acid sequence is introduced into that genome). As used herein, chromosomal and native and endogenous refer to genetic material of the host microorganism.

As used herein, the term “gene disruption,” or grammatical equivalents thereof (and including “to disrupt enzymatic function,” “disruption of enzymatic function,” and the like), is intended to mean a genetic modification to a microorganism that renders the encoded gene product as having a reduced polypeptide activity compared with polypeptide activity in or from a microorganism cell not so modified. The genetic modification can be, for example, deletion of the entire gene, deletion or other modification of a regulatory sequence required for transcription or translation, deletion of a portion of the gene which results in a truncated gene product (e.g., enzyme) or by any of various mutation strategies that reduces activity (including to no detectable activity level) the encoded gene product. A disruption may broadly include a deletion of all or part of the nucleic acid sequence encoding the enzyme, and also includes, but is not limited to other types of genetic modifications, e.g., introduction of stop codons, frame shift mutations, introduction or removal of portions of the gene, and introduction of a degradation signal, those genetic modifications affecting mRNA transcription levels and/or stability, and altering the promoter or repressor upstream of the gene encoding the enzyme.

Bio-production, Micro-fermentation (microfermentation) or Fermentation, as used herein, may be aerobic, microaerobic, or anaerobic.

When the genetic modification of a gene product, i.e., an enzyme, is referred to herein, including the claims, it is understood that the genetic modification is of a nucleic acid sequence, such as or including the gene, that normally encodes the stated gene product, i.e., the enzyme.

As used herein, the term “metabolic flux” and the like refers to changes in metabolism that lead to changes in product and/or byproduct formation, including production rates, production titers and production yields from a given substrate.

Species and other phylogenic identifications are according to the classification known to a person skilled in the art of microbiology.

Enzymes are listed here within, with reference to a UniProt identification number, which would be well known to one skilled in the art. The UniProt database can be accessed at http://www.UniProt.org/. When the genetic modification of a gene product, i.e., an enzyme, is referred to herein, including the claims, it is understood that the genetic modification is of a nucleic acid sequence, such as or including the gene, that normally encodes the stated gene product, i.e., the enzyme.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The meaning of abbreviations is as follows: “C” means Celsius or degrees Celsius, as is clear from its usage, DCW means dry cell weight, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)”, “g” means gram(s), “μg” or “ug” means microgram(s) and “ng” means nanogram(s), “PCR” means polymerase chain reaction, “OD” means optical density, “OD₆₀₀” means the optical density measured at a photon wavelength of 600 nm, “kDa” means kilodaltons, “g” means the gravitation constant, “bp” means base pair(s), “kbp” means kilobase pair(s), “% w/v” means weight/volume percent, “% v/v” means volume/volume percent, “IPTG” means isopropyl-μ-D-thiogalactopyranoiside, “aTc” means anhydrotetracycline, “RBS” means ribosome binding site, “rpm” means revolutions per minute, “HPLC” means high performance liquid chromatography, and “GC” means gas chromatography.

### Microorganisms

Features as described and claimed herein may be provided in a microorganism selected from the listing herein, or another suitable microorganism, that also comprises one or more natural, introduced, or enhanced product bio-production pathways. Thus, in some embodiments the microorganism(s) comprise an endogenous product production pathway (which may, in some such embodiments, be enhanced), whereas in other embodiments the microorganism does not comprise an endogenous product production pathway.

More particularly, based on the various criteria described herein, suitable microbial hosts for the bio-production of a chemical product generally may include, but are not limited to the organisms described herein.

The host microorganism or the source microorganism for any gene or protein described here may be selected from the following list of microorgansims: Citrobacter, Enterobacter, Clostridium, Klebsiella, Aerobacter, Lactobacillus, Aspergillus, Saccharomyces, Schizosaccharomyces, Zygosaccharomyces, Pichia, Kluyveromyces, Candida, Hansenula, Debaryomyces, Mucor, Torulopsis, Methylobacter, Escherichia, Salmonella, Bacillus, Streptomyces, and Pseudomonas. In some aspects the host microorganism is an E. coli microorganism.

### Genetic Modifications, Nucleotide Sequences, and Amino Acid Sequences

Embodiments of the present invention may result from introduction of an expression vector into a host microorganism, wherein the expression vector contains a nucleic acid sequence coding for an enzyme that is, or is not, normally found in a host microorganism.

The ability to genetically modify a host cell is essential for the production of any genetically modified (recombinant) microorganism. The mode of gene transfer technology may be by electroporation, conjugation, transduction, or natural transformation. A broad range of host conjugative plasmids and drug resistance markers are available. The cloning vectors are tailored to the host organisms based on the nature of antibiotic resistance markers that can function in that host. Also, as disclosed herein, a genetically modified (recombinant) microorganism may comprise modifications other than via plasmid introduction, including modifications to its genomic DNA.

More generally, nucleic acid constructs can be prepared comprising an isolated polynucleotide encoding a polypeptide having enzyme activity operably linked to one or more (several) control sequences that direct the expression of the coding sequence in a microorganism, such as E. coli, under conditions compatible with the control sequences. The isolated polynucleotide may be manipulated to provide for expression of the polypeptide. Manipulation of the polynucleotide's sequence prior to its insertion into a vector may be desirable or necessary depending on the expression vector. The techniques for modifying polynucleotide sequences utilizing recombinant DNA methods are well established in the art.

The control sequence may be an appropriate promoter sequence, a nucleotide sequence that is recognized by a host cell for expression of a polynucleotide encoding a polypeptide of the present invention. The promoter sequence may contain transcriptional control sequences that mediate the expression of the polypeptide. The promoter may be any nucleotide sequence that shows transcriptional activity in the host cell of choice including mutant, truncated, and hybrid promoters, and may be obtained from genes encoding extracellular or intracellular polypeptides either homologous or heterologous to the host cell. The techniques for modifying and utilizing recombinant DNA promoter sequences are well established in the art.

For various embodiments of the invention the genetic manipulations may include a manipulation directed to change regulation of, and therefore ultimate activity of, an enzyme or enzymatic activity of an enzyme identified in any of the respective pathways. Such genetic modifications may be directed to transcriptional, translational, and post-translational modifications that result in a change of enzyme activity and/or selectivity under selected culture conditions. Genetic manipulation of nucleic acid sequences may increase copy number and/or comprise use of mutants of an enzyme related to product production. Specific methodologies and approaches to achieve such genetic modification are well known to one skilled in the art.

In various aspects, to function more efficiently, a microorganism may comprise one or more synthetic metabolic valves, composed of enzymes targeted for controlled proteolysis, expression silencing or a combination of both controlled proteolysis and expression silencing. For all nucleic acid and amino acid sequences provided herein, it is appreciated that conservatively modified variants of these sequences are included, and are within the scope of the invention in its various embodiments. Functionally equivalent nucleic acid and amino acid sequences (functional variants), which may include conservatively modified variants as well as more extensively varied sequences, which are well within the skill of the person of ordinary skill in the art, and microorganisms comprising these, also are within the scope of various embodiments of the invention, as are methods and systems comprising such sequences and/or microorganisms.

Accordingly, as described in various sections above, some compositions, methods and systems of the present invention comprise providing a genetically modified microorganism that comprises both a production pathway to make a desired product from a central intermediate in combination with synthetic metabolic valves to redistribute flux.

Aspects of the invention also regard provision of multiple genetic modifications to improve microorganism overall effectiveness in converting a selected carbon source into a selected product. Particular combinations are shown, such as in the Examples, to increase specific productivity, volumetric productivity, titer and yield substantially over more basic combinations of genetic modifications.

### Synthetic Metabolic Valves

Use of synthetic metabolic valves allows for simpler models of metabolic fluxes and physiological demands during a production phase, turning a growing cell into a stationary phase biocatalyst. These synthetic metabolic valves can be used to turn off essential genes and redirect carbon, electrons and energy flux to product formation in a multi-stage fermentation process. One or more of the following provides the described synthetic valves: 1) transcriptional gene silencing or repression technologies in combination with 2) inducible and selective enzyme degradation and 3) nutrient limitation to induce a stationary or non-dividing cellular state. SMVs are generalizable to any pathway and microbial host. These synthetic metabolic valves allow for novel rapid metabolic engineering strategies useful for the production of renewable chemicals and fuels and any product that can be produced via whole cell catalysis.

In particular, the invention describes the construction of synthetic metabolic valves comprising one or more or a combination of the following: controlled gene silencing and controlled proteolysis. It is appreciated that one well skilled in the art is aware of several methodologies for gene silencing and controlled proteolysis.

### Gene Silencing

In particular, the invention describes the use of controlled gene silencing to provide the control over metabolic fluxes in controlled multi-stage fermentation processes. There are several methodologies known in the art for controlled gene silencing, including but not limited to mRNA silencing or RNA interference, silencing via transcriptional repressors and CRISPR interference. Methodologies and mechanisms for RNA interference are taught by Agrawal et al. “RNA Interference: Biology, Mechanism, and Applications” Microbiology and Molecular Biology Reviews, December 2003; 67(4) p 657-685. DOI: 10.1128/MMBR.67.657-685.2003. Methodologies and mechanisms for CRISRPR interference are taught by Qi et al. “Repurposing CRISPR as an RNA-guided platform for sequence-specific control of gene expression” Cell February 2013; 152(5) p 1173-1183. DOI: 10.1016/j.cell.2013.02.022. In addition, methodologies and mechanisms for CRISRPR interference using the native E. coli CASCADE system are taught by Luo et al. “Repurposing endogenous type I CRISPR-Cas systems for programmable gene repression” NAR. October 2014; DOI: 10.1093. In additional numerous transcriptional repressor systems are well known in the art and can be used to turn off gene expression.

### Controlled Proteolysis

In particular, the invention describes the use of controlled protein degradation or proteolysis to provide the control over metabolic fluxes in controlled multi-stage fermentation processes. There are several methodologies known in the art for controlled protein degradation, including but not limited to targeted protein cleavage by a specific protease and controlled targeting of proteins for degradation by specific peptide tags. Systems for the use of the E. coli clpXP protease for controlled protein degradation are taught by McGinness et al, “Engineering controllable protein degradation”, Mol Cell. June 2006; 22(5) p 701-707. This methodology relies upon adding a specific C-terminal peptide tag such as a DAS4 (or DAS+4) tag. Proteins with this tag are not degraded by the clpXP protease until the specificity enhancing chaperone sspB is expressed. sspB induces degradation of DAS4 tagged proteins by the clpXP protease. In additional numerous site specific protease systems are well known in the art. Proteins can be engineered to contain a specific target site of a given protease and then cleaved after the controlled expression of the protease. In some embodiments, the cleavage can be expected lead to protein inactivation or degradation. For example Schmidt et al(“ClpS is the recognition component for Escherichia coli substrates of the N-end rule degradation pathway” Molecular Microbiology March 2009. 72(2), 506-517. doi:10.1111), teaches that an N-terminal sequence can be added to a protein of interest in providing clpS dependent clpAP degradation. In addition, this sequence can further be masked by an additional N-terminal sequence, which can be controllable cleaved such as by a ULP hydrolase. This allows for controlled N-rule degradation dependent on hydrolase expression. It is therefore possible to tag proteins for controlled proteolysis either at the N-terminus or C-terminus. The preference of using an N-terminal vs. C-terminal tag will largely depend on whether either tag affects protein function prior to the controlled onset of degradation.

The invention describes the use of controlled protein degradation or proteolysis to provide the control over metabolic fluxes in controlled multi-stage fermentation processes, in E. coli. There are several methodologies known in the art for controlled protein degradation in other microbial hosts, including a wide range of gram-negative as well as gram-positive bacteria, yeast and even archaea. In particular, systems for controlled proteolysis can be transferred from a native microbial host and used in a non-native host. For example Grilly et al, “A synthetic gene network for tuning protein degradation in Saccharomyces cerevisiae” Molecular Systems Biology 3, Article 127. doi:10.1038, teaches the expression and use of the E. coli clpXP protease in the yeast Saccharomyces cerevisiae. Such approaches can be used to transfer the methodology for synthetic metabolic valves to any genetically tractable host.

### Synthetic Metabolic Valve Control

In particular the invention describes the use of synthetic metabolic valves to control metabolic fluxes in multi-stage fermentation processes. There are numerous methodologies known in the art to induce expression that can be used at the transition between stages in multi-stage fermentations. These include but are not limited to artificial chemical inducers including: tetracycline, anhydrotetracycline, lactose, IPTG (isopropyl-beta-D-1-thiogalactopyranoside), arabinose, raffinose, tryptophan and numerous others. Systems linking the use of these well known inducers to the control of gene expression silencing and/or controlled proteolysis can be integrated into genetically modified microbial systems to control the transition between growth and production phases in multi-stage fermentation processes.

In addition, it may be desirable to control the transition between growth and production in multi-stage fermentations by the depletion of one or more limiting nutrients that are consumed during growth. Limiting nutrients can include but are not limited to: phosphate, nitrogen, sulfur and magnesium. Natural gene expression systems that respond to these nutrient limitations can be used to operably link the control of gene expression silencing and/or controlled proteolysis to the transition between growth and production phases in multi-stage fermentation processes.

In various embodiments, the invention includes a culture system comprising a carbon source in an aqueous medium and a genetically modified microorganism according to any one of claims herein, wherein said genetically modified organism is present in an amount selected from greater than 0.05 gDCW/L, 0.1 gDCW/L, greater than 1 gDCW/L, greater than 5 gDCW/L, greater than 10 gDCW/L, greater than 15 gDCW/L or greater than 20 gDCW/L, such as when the volume of the aqueous medium is selected from greater than 5 mL, greater than 100 mL, greater than 0.5 L, greater than 1 L, greater than 2 L, greater than 10 L, greater than 250 L, greater than 1000 L, greater than 10,000 L, greater than 50,000 L, greater than 100,000 L or greater than 200,000 L, and such as when the volume of the aqueous medium is greater than 250 L and contained within a steel vessel.

### Overview of Invention Aspects

In one aspect of the invention, a genetically modified microorganism host comprising: inducible expression of a production protein, inducible regulation of at least one first protease of the genetically modified microorganism is described. The genetically modified microorganism is characterized by, after induction, the level of at least one first protease is reduced and the production protein is produced.

In one aspect, the protease is a conditionally essential protease.

In another aspect, the level of the least one first protease is reduced through proteolysis. The level referring to a detectable amount of the protease enzyme or enzymatic activity that is detectable.

In another aspect, the level of the least one first protease is reduced through gene silencing.

In another aspect, the level of the least one first protease is reduced through a combination of proteolysis and gene silencing.

In another aspect, the genetically modified microorganism may be additionally genetically modified to delete from the chromosome of the microorganism at least one first protease.

In another aspect, the genetically modified microorganism is genetically modified to delete from the chromosome of the microorganism a protease gene selected from the group consisting of lon, hslUV, ClpXP, ClpAP, pepP, dcp, elaD, hyaD, hybD, pepD, pepQ, pepT, pqqL, prlC, ptrB, tldD, ycaL, loiP, ypdF, degS, glpG, htpX, gspO, pppA, sohB, sppA, degP, degQ, iap, nlpC, prc, ptrA, tesA, yafL, ydgD, and ompT.

In another aspect, the genetically modified microorganism is an E. coli microorganism.

In another aspect, the genetically modified microorganism the protease to be reducted is selected from the group consisting of: ftsH protease, lipoprotein signal peptidase (lspA), methionine aminopeptidase, lon, ClpXP, ClpAP, hslUV, lepB, and rseP.

In another aspect, the genetically modified microorganism is additionally genetically modified to reduce the levels of at least one additional second protease.

In another aspect, the levels of the second protease may be reduced through proteolysis.

In another aspect, the levels of the second protease may be reduced through gene silencing.

In another aspect, the levels of the second protease may be reduced through a combination of proteolysis and gene silencing.

In another aspect, a second protease to be regulated by the genetically modified microorganism is encoded by a gene selected from the group consisting of: lon, hslUV, ClpXP, ClpAP, pepP, dcp, elaD, hyaD, hybD, pepD, pepQ, pepT, pqqL, prlC, ptrB, tldD, ycaL, loiP, ypdF, degS, glpG, htpX, gspO, pppA, sohB, sppA, degP, degQ, iap, nlpC, prc, ptrA, tesA, yafL, ydgD, and ompT.

### Disclosed Embodiments are Non-Limiting

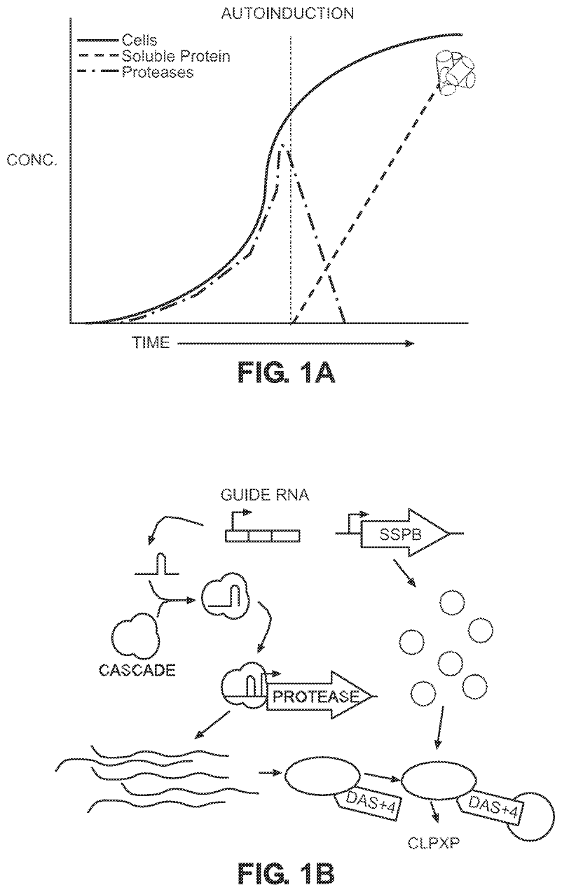

While various embodiments of the present invention have been shown and described herein, it is emphasized that such embodiments are provided by way of example only. Numerous variations, changes and substitutions may be made without departing from the invention herein in its various embodiments. Specifically, and for whatever reason, for any grouping of compounds, nucleic acid sequences, polypeptides including specific proteins including functional enzymes, metabolic pathway enzymes or intermediates, elements, or other compositions, or concentrations stated or otherwise presented herein in a list, table, or other grouping (such as metabolic pathway enzymes shown in a FIG. 1 unless clearly stated otherwise, it is intended that each such grouping provides the basis for and serves to identify various subset embodiments, the subset embodiments in their broadest scope comprising every subset of such grouping by exclusion of one or more members (or subsets) of the respective stated grouping. Moreover, when any range is described herein, unless clearly stated otherwise, that range includes all values therein and all sub-ranges therein.

Also, and more generally, in accordance with disclosures, discussions, examples and embodiments herein, there may be employed conventional molecular biology, cellular biology, microbiology, and recombinant DNA techniques within the skill of the art. Such techniques are explained fully in the literature. See, e.g., Sambrook and Russell, “Molecular Cloning: A Laboratory Manual,” Third Edition 2001 (volumes 1-3), Cold Spring Harbor Laboratory Press, Cold Spring Harbor, N.Y.; Animal Cell Culture, R. I. Freshney, ed., 1986. These published resources are incorporated by reference herein.

The following published resources are incorporated by reference herein for description useful in conjunction with the invention described herein, for example, methods of industrial bio-production of chemical product(s) from sugar sources, and also industrial systems that may be used to achieve such conversion (Biochemical Engineering Fundamentals, 2nd Ed. J. E. Bailey and D. F. 011 is, McGraw Hill, New York, 1986, e.g. Chapter 9, pages 533-657 for biological reactor design; Unit Operations of Chemical Engineering, 5^{th} Ed., W. L. McCabe et al., McGraw Hill, New York 1993, e.g., for process and separation technologies analyses; Equilibrium Staged Separations, P. C. Wankat, Prentice Hall, Englewood Cliffs, N.J. USA, 1988, e.g., for separation technologies teachings).

All publications, patents, and patent applications mentioned in this specification are entirely incorporated by reference.

### EXAMPLES

The examples herein provide some examples, not meant to be limiting. All reagents, unless otherwise indicated, are obtained commercially. Species and other phylogenic identifications are according to the classification known to a person skilled in the art of microbiology, molecular biology and biochemistry.

### Example 1: Two-Stage Synthetic Metabolic Valves

Referring now to FIG. 1A-B, we developed synthetic metabolic valves (SMVs), which are capable of the dynamic reduction of protein levels in a two-stage process. These SMVs rely on controlled proteolysis or CRISPRi/Cascade based gene silencing or both proteolysis and silencing in combination to reduce levels of key metabolic enzymes. Cell growth and dynamic metabolic control is implemented using phosphate depletion, for example, as an environmental trigger, in autoinduction broth as previously described.

More specifically, in FIG. 1, two-stage dynamic control over host machinery such as proteases. a) Time course of 2-stage dynamic control, cells (black) line grow until a limiting nutrient is depleted, in this case phosphate. As phosphate is depleted autoinduction of recombinant protein expression (green) occurs in parallel with reduction in key enzymes such as housekeeping proteases. b) Dynamic turnover of housekeeping proteases can be achieved through i) CRISPR mediated gene silencing and controlled proteolysis alone and or in combination. gRNA(s) are induced upon nutrient depletion and along with the native nuclease deficient Cascade complex of E. coli (with a deletion of cas3 the nuclease component of the Cascade complex) bind to targeted promoters reducing gene expression. In parallel, the protein of interest for turnover is modified to have a C-terminal degron tag. Upon nutrient depletion induction of the sspB gene produces a chaperone binding to the degron tag and targeting the protein for clpXP mediated proteolysis.

It has recently been shown that deleting the cas3 gene and expressing guide RNAs can lead to gene silencing. Targeted proteolysis was controlled by linking the expression of the chaperone sspB to phosphate deprivation. SspB, when induced, binds to C-terminal DAS+4 (DAS4) peptide tags on any target protein and causes degradation by the clpXP protease of E. coli (FIG. 1B). These efforts resulted in an E. coli host strain (DLF_Z0025) capable of wild type growth rates, high biomass yields, and the low phosphate induction of proteolytic and CRISPR/Cascade machinery.

The following Table 1 provides a complete list of strains and plasmids used in this study:

TABLE 1
Plasmids and strain used in this study

| Plasmid | Insert | promoter | ori | Res | Addgene | Source |
|---|---|---|---|---|---|---|
| pSMART-HC-Kan | None | None | colE1 | Kan | NA | Lucigen |
| pHCKan-yibDp-GFPuv | GFPuv | yibDp | colE1 | Kan | 127078 | Menacho- |
| | | | | | | Melgar, et |
| | | | | | | al |
| pBT1-proDp-mCherry | mCherry | proD | BBR1 | Amp | TBD | This study |
| pTKhc-yibDp-GFP-β20cp6 | GFP-β20-cp6 | yibD | colE1 | Kan | 127060 | This study |
| pTKhc-yibDp-GFP-cp6 | GFP-cp6 | yibD | colE1 | Kan | 134938 | This study |
| pCASCADE-EV | None | ugpBp1 | p15a | Cm | 65821 | This study |
| pCASCADE-proD | proDp gRNA | ugpBp1 | p15a | Cm | 65820 | This study |
| pCASCADE-lon | lon | ugpBp1 | p15a | Cm | TBD | This study |
| pCASCADE-ftsHp1 | ftsH | ugpBp1 | p15a | Cm | TBD | This study |
| pCASCADE-ftsHp2 | ftsH | ugpBp1 | p15a | Cm | TBD | This study |
| pCASCADE-ftsHp3 | ftsH | ugpBp1 | p15a | Cm | TBD | This study |
| pCASCADE-ftsHp4 | ftsH | ugpBp1 | p15a | Cm | TBD | This study |

Strains used in this study

| Strain | Genotype | Source |
|---|---|---|
| DLF_R002 | F-, λ-, Δ(araD-araB)567, lacZ4787(del)(::rrnB-3), rph-1, Δ(rhaD- | Menacho- |
| | rhaB)568, hsdR514, ΔackA-pta, ΔpoxB, ΔpflB, ΔldhA, ΔadhE, | Melgar, et |
| | ΔiclR, ΔarcA | al |
| DLF_Z001 | DLF_R002, ΔsspB::frt | This study |
| DLF_Z002 | DLF_R002, Δcas3:: pro-casA | This study |
| DLF_Z0025 | DLF_Z001, Δcas3:: ugpBp-sspB-pro-casA | This study |
| DLF_R0010 | DLF_Z001, Δcas3:: ugpBp-sspB-pro-casA, lon-DAS4:zeoR | This study |
| DLF_R0011 | DLF_Z001, Δcas3:: ugpBp-sspB-pro-casA, ftsH-DAS4:ampR | This study |
| DLF_R0012 | DLF_Z001, Δcas3:: ugpBp-sspB-pro-casA, ftsH-DAS4:ampR, | This study |
| | ΔhslUV::tetR | |

Res—resistance marker, ori—origin of replication,, Cm—chloramphenicol, Kan—kanamycin, Amp—ampicillin, tet—tetracycline, zeo—zeocin

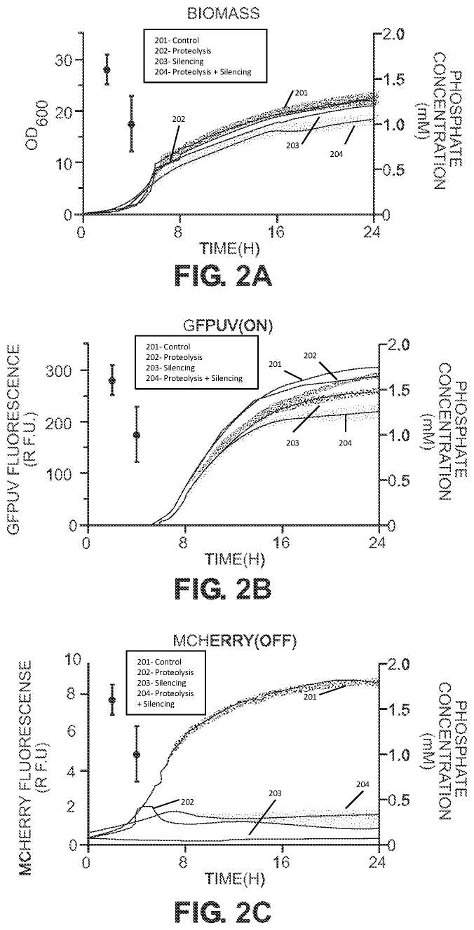

Referring now to FIG. 2, two-stage dynamic control over fluorescent proteins in autoinduction broth. A series of DLF_Z0025 derivatives with autoinducible GFP were evaluated in autoinduction broth for 2-stage dynamic control. These included: a no mCherry control (Empty, gray line), an mCherry with an empty gRNA silencing vector (Cont. black line), an mCherry with a degron tag (Proteolysis, purple line), a gRNA silencing the mCherry promoter (Silencing, green line) as well as an mCherry with a degron tag and a gRNA silencing the mCherry promoter (S+P, red line). a) Growth, b) GFP expression and c) mCherry levels over time.

Using this system, as FIG. 2 demonstrates, protein levels can be controlled in a 2-stage processes, as exemplified by turning “ON’ GFP and “OFF” mCherry fluorescent proteins with phosphate depletion in autoinduction broth. While, in this case, the combination of gene silencing with proteolysis results in the largest rates of protein degradation (FIG. 2c), the impact of each approach and specific decay rates, will vary dependent on the target gene/enzyme and its specific natural turnover rates and expression levels.

### Example 2: Dynamic Control over Lon Protease

With the successful demonstration of dynamic protein turnover in a 2-stage process with autoinduction, we turned to investigate the dynamic control of the key housekeeping protease Lon. Although not strictly essential, Lon has important regulatory roles in cell division, capsule synthesis, recovery from the SOS response and general stress tolerance and protein quality control, making complete deletions nonideal.

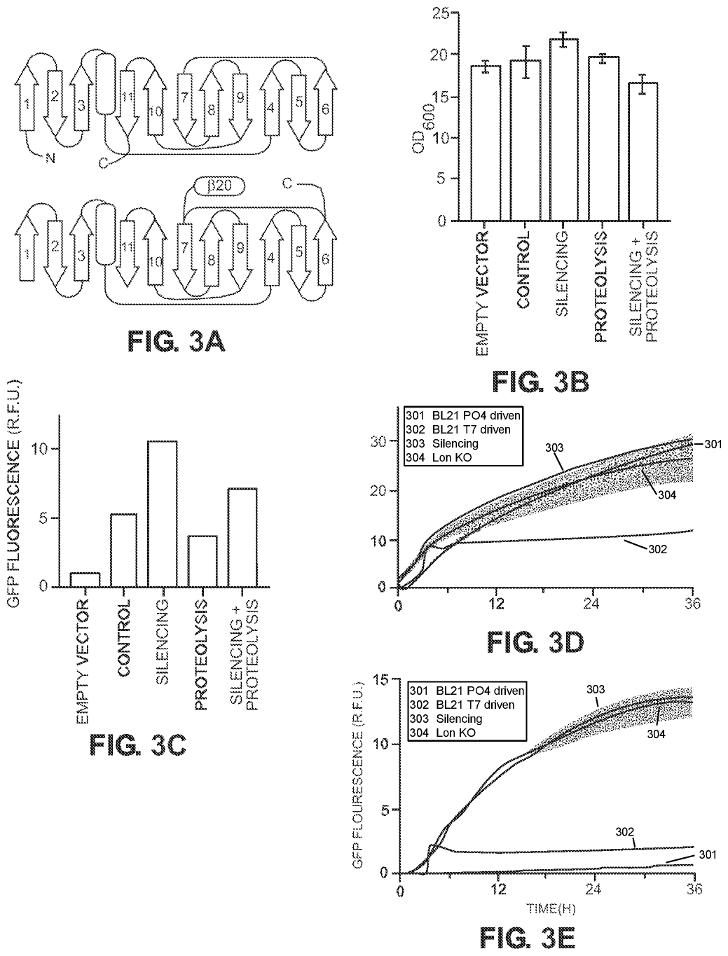

Referring now to FIG. 3, 2-stage dynamic control over Lon protease activity. (a) a schematic of superfolder GFP (top), and a schematic of the Beta-20 tagged circularly permuted GFP (β20-cp6-GFP) (bottom), which is a substrate for the Lon protease. Reduction of Lon protease levels was evaluated in a series of DLF_Z0025 strains by measuring fluorescence of the Lon substrate after 24 hours, we show (b) biomass levels and (c) fluorescence values. Then, we compared Lon substrate protein levels and dynamics of lon silencing (grey) to the BL21(DE3) strain bearing a plasmid for the induction of the GFP Lon substrate under phosphate limitation (red) and T7-driven induction (blue), as well as a lon knock-out (blue); for which (d) biomass levels and (e) fluorescence levels are shown.

Strains were constructed with either modifications to induce proteolysis or gene silencing alone or in combination. These strains were then evaluated using a previously reported fluorescent Lon substrate, β20-cp6-GFP, as shown in FIG. 3b. 37 In this case, protein (fluorescence) should be increased when Lon activity is decreased, as Lon degrades the protein being induced. Results are given in FIG. 3. Silencing of lon expression had the largest impact of protein levels, with a ˜2.5 fold improvement. Interestingly, unlike the case of mCherry (FIG. 2), proteolysis had a minimal impact of Lon activity and the combination of silencing and proteolysis was not an improvement over silencing alone (FIG. 3d). This could be due to the DAS4 tag altering the natural turnover, or activity of Lon, not being accessible to the sspB chaperone or altered transcript stability and basal expression levels with the chromosomal modifications adding the DAS4 degron tag. Alternatively, perhaps basal Lon activity is required for optimal expression and induction. In any event, This requires further investigation. As silencing alone had a significant impact on the expression levels of Lon substrate, future work leveraged silencing to reduce Lon activity.

### Example 3: Dynamic Control Over FtsH Protease

We next turned to the dynamic control over another key essential, membrane anchored protease in E. coli, FtsH, which also serves regulatory as well as protein quality control functions. 38 FtsH is essential in E. coli, due to its regulation of lipid A synthesis and deletions can only be made with key suppressor mutations in the fabZ gene. As in the case of lon, we engineered strains to introduce a C-terminal degron tag to FtsH, however for the ftsH gene, there are four known or anticipated promoters, and as a result 4 different silencing gRNAs were constructed (Table 1). These strains were then evaluated for changes in ftsH activity by evaluated the expression of miraculin, a small plant protein previously shown to be a substrate for FtsH.

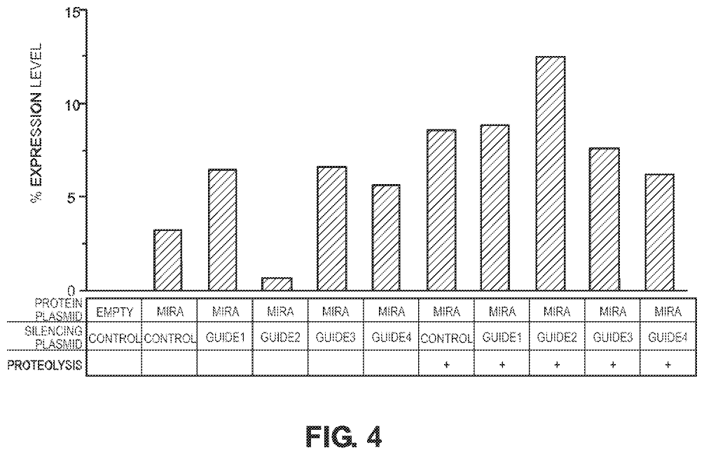

Referring now to FIG. 4, 2-stage dynamic control over FtsH protease activity. 2-stage dynamic control over the autoinducible miraculin substrate was evaluated in a series of DLF_Z0025 derivatives. These included: DLF_Z0025 (no proteolysis), DLF_R0011 a strain with a DAS4 degron tag on FtsH (+Proteolysis), an empty vector (no miraculin) or a plasmid containing the mirA gene encoding miraculin and plasmids with either an empty gRNA or silencing gRNA targeting each of the four ftsH gene promoters.

Results are given in FIG. 4 below. In the case of FtsH, both silencing and proteolytic control improved micraculin expression, with silencing of the second ftsH promoter combined with proteolytic degradation of ftsH, having a four fold improvement over the control and reaching expression levels of ˜12%.

### Example 4: Improved Expression of “Difficult to Express” Proteins

With successful reduction in FtsH and Lon activity in stationary phase, as demonstrated by increased expression of their respective substrates, we turned to evaluate the broader applicability of this approach to improve the expression to difficult to express proteins. First we constructed a strain with a ftsH degron tag with a deletion in the nonessential HslUV protease, to further reduce housekeeping proteolysis, strain DLF_R0012 (Table 1). The other two major housekeeping proteases in E. coli are the ClpAP and ClpXP proteases, of which the ClpP subunit is essential for our controlled proteolysis, and so these were not manipulated in this study. In addition, we constructed a combined silencing gRNA array to silence both lon and the second promoter of ftsH.

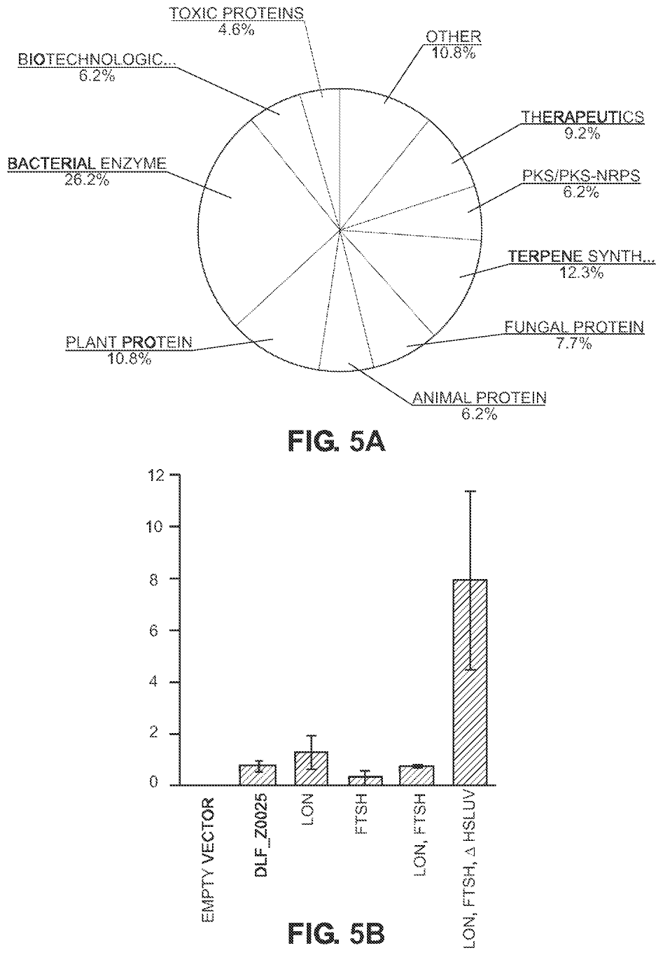

Referring now to FIG. 5, improved protein expression with dynamic control over host proteases is shown. A) A group of 60 diverse difficult to express proteins, including 6 therapeutics, 4 PKS or PKS-NRPS enzymes, 8 terpene synthases, 5 fungal proteins, 4 animal proteins, 7 plant proteins (non terpene synthases), 17 bacterial enzymes, 3 known toxic proteins and 7 additional proteins. B) 2-stage dynamic control over protease activity improves expression of the 220 kDa PKS-NRPS, salinosporamide synthase from S. tropica. Expression of the 220 kDa protein, in the control lacking dynamic control over proteases (DLF_Z0025) was compared to strains with dynamic control in Lon, FtsH, both Lon and FtsH as well as the deletion of the third major housekeeping protease, HslUV.

With these strains we sought to evaluate the expression of over 60 “difficult” to express proteins as illustrated in FIG. 5a, comprising a group of bacterial, fungal, plant, animal proteins, as well as known toxic proteins, natural product PKS or PKS/NRPS enzymes, cytochrome P450s, terpene synthases, therapeutic molecules, and proteins of biotechnological interest such as Cas9. Each of these proteins was cloned behind the autoinducible yibDp promoter. In addition in several cases pET (T7) based expression constructs were constructed or obtained to enable a head to head evaluation with standard BL21(DE3) production. Refer to Supplemental Table S2 for protein and construct details. While final results for most of these studies are pending, results for a large difficult to express 220 kDa PKS/NRPS, salinosporamide synthase encoded by the salABE genes of Salinospora tropica are given in FIG. 5b below. Dynamic control over proteases resulted in significant increases in expression to ˜12% of total cell protein.

1. A genetically modified microorganism host comprising:
inducible expression of a production protein,
inducible regulation of at least one first protease of the genetically modified microorganism,
wherein, after induction, the level of at least one first protease is reduced and the production protein is produced.

2. The genetically modified microorganism of claim 1, wherein the first protease is a conditionally essential protease.

3. The genetically modified microorganism of claim 1, wherein the level of the least one first protease is reduced through proteolysis.

4. The genetically modified microorganism of claim 1, wherein the level of the at least one first protease is reduced through gene silencing.

5. The genetically modified microorganism of claim 1, wherein the level of the at least one first protease is reduced through a combination of proteolysis and gene silencing.

6. The genetically modified microorganism of claim 1, wherein the microorganism is additionally genetically modified to delete from the chromosome of the microorganism.

7. The genetically modified microorganism of claim 1, wherein the microorganism is additionally genetically modified to delete from the chromosome of the microorganism a protease gene selected from the group consisting of lon, hslUV, ClpXP, ClpAP, pepP, dcp, elaD, hyaD, hybD, pepD, pepQ, pepT, pqqL, prlC, ptrB, tldD, ycaL, loiP, ypdF, degS, glpG, htpX, gspO, pppA, sohB, sppA, degP, degQ, iap, nlpC, prc, ptrA, tesA, yafL, ydgD, and ompT.

8. The genetically modified microorganism of claim 1, wherein the microorganism is an E. coli microorganism.

9. The genetically modified microorganism of claim 1, wherein the protease is selected from the group consisting of: ftsH protease, lipoprotein signal peptidase (lspA), methionine aminopeptidase, lon, ClpXP, ClpAP, hslUV, lepB, and rseP.

10. The genetically modified microorganism of claim 1, wherein the microorganism is additionally genetically modified to reduce the levels of at least one additional second protease.

11. The genetically modified microorganism of claim 10, wherein the levels of the second protease is reduced through proteolysis.

12. The genetically modified microorganism of claim 10, wherein the levels of the second protease is reduced through gene silencing.

13. The genetically modified microorganism of claim 10, wherein the levels of the second protease is reduced through a combination of proteolysis and gene silencing.

14. The genetically modified microorganism of claim 10, wherein the second protease is encoded by a genes that is deleted, the gene being selected from the group consisting of: lon, hslUV, ClpXP, ClpAP, pepP, dcp, elaD, hyaD, hybD, pepD, pepQ, pepT, pqqL, prlC, ptrB, tldD, ycaL, loiP, ypdF, degS, glpG, htpX, gspO, pppA, sohB, sppA, degP, degQ, iap, nlpC, prc, ptrA, tesA, yafL, ydgD, and ompT.
